(19) United States
(12) Patent Application Publication
(10) Pub. No.: US 2018/0086694 A1
(43) Pub. Date: Mar. 29, 2018

(54) DISINFECTING COMPOSITIONS HAVING IMPROVED ANTIMICROBIAL EFFICACY

(71) Applicants: Michael Lisle Howell, Mason, OH (US); Zsolt Istvan Hertelendy, Mason, OH (US); Joseph Paul Thomas, Edgewood, KY (US)

(72) Inventors: Michael Lisle Howell, Mason, OH (US); Zsolt Istvan Hertelendy, Mason, OH (US); Joseph Paul Thomas, Edgewood, KY (US)

(21) Appl. No.: 15/712,666

(22) Filed: Sep. 22, 2017

Related U.S. Application Data
(60) Provisional application No. 62/399,057, filed on Sep. 23, 2016.
(60) Provisional application No. 62/422,481, filed on Nov. 15, 2016.

(51) Int. Cl. C07C 229/16; A61K 31/132; A61K 31/045; A01N 31/08; A61L 2/00

(52) U.S. Cl. CPC C07C 229/16; A61K 31/132; A61K 31/045; C12N 1/20; A61L 2/0088; C12N 2760/16163; A01N 31/08

(57) ABSTRACT

Disinfecting compositions effective against multiple bacterial, fungal or viral organisms are described herein. Personal or consumer care products as well as clinical disinfectant products including the disinfecting compositions are also described.

### CROSS-REFERENCE TO RELATED APPLICATIONS

This application claims the priority benefit of U.S. Provisional Patent Application Ser. No. 62/399,057, “Disinfecting Compositions Having Improved Antimicrobial Efficacy,” filed Sep. 23, 2016, and U.S. Provisional Patent Application Ser. No. 62/422,481, “Disinfecting Compositions Having Improved Antimicrobial Efficacy,” filed Nov. 15, 2016, both of which are hereby expressly incorporated by reference in their entirety.

### TECHNICAL FIELD

The present disclosure generally relates to disinfecting compositions. Specifically, the present disclosure relates to new compositions providing improved antimicrobial efficacy, as well as health and consumer care products including the same.

### BACKGROUND

Pathogenic microorganisms such as bacteria, viruses, and fungi are responsible for a host of human diseases, ranging from minor upper and lower respiratory tract infections to potentially fatal infections. Major progress has been made in combating diseases caused by pathogenic microorganisms with pharmaceutical and non-pharmaceutical agents having antimicrobial effect. For example, some chemical-based agents are used for external treatment (e.g., on a hard surface) to prevent contamination and transmission to humans and others are used to disinfect skin in the clinical and non-clinical settings. While agents have been developed that are generally effective against various pathogens, there is increasing evidence that the use of such agents has certain limitations that warrant concern. Specifically, certain strains of pathogenic microorganisms have become increasingly resistant to one or more antibiotics, thereby rendering the standard courses of treatment ineffective. As a result, higher doses of antimicrobial treatments are increasingly required to achieve efficacy, which result in undesirable side effects and toxicity to both humans and the environment. On the other hand, many disinfectants do not persist in antimicrobial activity once applied on surfaces or skin, which render them useless in situations where frequent application is difficult.

There is therefore a need for new compositions with improved antimicrobial efficacy to prevent contamination or to disinfect contaminated surfaces or skin, and that retains antimicrobial activity for several days after application.

### SUMMARY

In one embodiment, a disinfecting composition includes an antimicrobial agent. The composition includes from about 1.5% to about 2.0% by weight per volume of an ethylenediamine tetraacetic acid or salts thereof (EDTA); from about 4% to about 4.5% by weight per volume of a straight chain 1,2-alkanediol, wherein the straight chain 1,2-alkanediol includes 1,2-hexanediol; from about 0.15% to about 0.5% by weight per volume of a surfactant and from about 0.15% to about 0.5% by weight per volume of one of a biocidal terpenoid or an essential oil.

### DETAILED DESCRIPTION

In the following detailed description, reference is made to various embodiments a new disinfecting composition and use thereof. Sufficient detail is described herein to enable those skilled in the art to practice the various embodiments. Alternative embodiments may be utilized and structural, logical, and chemical changes may be made without departing from the scope of the present invention. The following detailed description is, therefore, not to be taken in a limiting sense, and the scope of the present invention is defined only by the appended claims and equivalents thereof.

Effective treatment for and prevention of bacterial, viral and fungal infections is necessary, particularly in settings like hospitals, clinics, medical research centers, nursing homes, schools and athletic facilities such as gymnasiums, health clubs, and spas. Hospitals, schools, athletic facilities and similar institutions are always looking for effective solutions to reduce or prevent the spread of ancillary infections such as Staphylococcus aureus (ATCC #6538). One such example is commonly known as methicillin-resistant Staphylococcus aureus, or MRSA. Outbreaks of MRSA or other bacterial infections are major concerns for medical facilities, schools, nursing homes, etc. To curb the likelihood of such unforeseen outbreaks, hospitals, schools and other institutions have implemented the use of disinfectants and training protocols to reduce such risks. However, such disinfectants do not necessarily offer the antimicrobial benefits or lasting effectiveness desired by these institutions. The present disclosure describes improved disinfectants containing antimicrobial agents and methods of using such disinfectants to achieve improved efficacy and safety profile, as well as persistent antimicrobial effect.

The disinfecting compositions described herein provide improved antimicrobial efficacy against multiple types of bacteria, including certain fungal and viral strains that are not commonly achieved by other traditional antimicrobial products. For example, according to certain embodiments, the disinfecting compositions described herein are effective with respect to both Staphylococcus aureus (ATCC #6538) and Pseudomonas aeruginosa (ATCC #15442).

The disinfecting compositions described herein in one embodiment can include an antimicrobial agent. The antimicrobial agent can include ethylenediamine tetraacetic acid or salts thereof (EDTA), a straight chain 1,2-alkanediol, such as 1,2-hexanediol, a surfactant, and one of biocidal terpenoid or source for a biocidal terpenoid, such as an essential oil.

In certain embodiments, an antimicrobial agent can have more than 1.5% by weight per volume of EDTA; in certain embodiments from about 1.5% to about 2.0% by weight per volume of EDTA; in certain embodiments from about 1.6% to about 1.9% by weight per volume of EDTA; and in certain embodiments from about 1.6% to about 1.8% by weight per volume of EDTA.

In certain embodiments, an antimicrobial agent can have from about 4% to about 4.5% by weight per volume of a straight chain 1,2-alkanediol, such as 1,2-hexanediol; and in certain embodiments about 4.25% by weight per volume of a straight chain 1,2-alkanediol, such as 1,2-hexanediol. In certain embodiments, a straight chain 1,2-alkanediol can have a chain length in the range of 5 to 10 carbon atoms. Such 1,2-alkanediols include, 1,2-pentanediol, 1,2-hexanediol, 1,2-heptanediol, 1,2-octanediol, 1,2-nonanediol, and 1,2-decanediol. In certain embodiments, the antimicrobial agent can include a mixture of straight chain 1,2-alkanediols, for example, 1,2-pentanediol and 1,2-hexanediol.

In certain embodiments, an antimicrobial agent can have from about 0.1% to about 0.5% by weight per volume of a surfactant; in certain embodiments from about 0.15% to about 0.4% by weight per volume of a surfactant; in certain embodiments from about 0.175% to about 0.3% by weight per volume of a surfactant; and in certain embodiments about 0.2% by weight per volume of a surfactant. The concentration of surfactant in a resulting disinfecting composition may be higher in other embodiments.

Surfactants can be selected from anionic, cationic, nonionic, or zwitterionic surfactants in various embodiments. Suitable surfactants can include a cationic salt of dodecyl sulfate (e.g., sodium dodecyl sulfate), a Triton® surfactant (e.g., Triton® X-100), tetradecyldimethyl(3-sulfopropyl)-ammonium hydroxide inner salt, or combinations thereof. In one example, the surfactant can be sodium dodecyl sulfate. Other suitable surfactants may also be included in certain embodiments.

In certain embodiments, an antimicrobial agent can include a biocidal terpenoid or an oil compound (such as an essential oil) that serves as a source for the biocidal terpenoid. In certain embodiments, an antimicrobial agent can have from about 0.1% to about 0.5% by weight per volume of a biocidal terpenoid or an essential oil; in certain embodiments from about 0.15% to about 0.4% by weight of a biocidal terpenoid or an essential oil; in certain embodiments from about 0.175% to about 0.3% of a biocidal terpenoid or an essential oil; and in certain embodiments about 0.2% by weight per volume of a biocidal terpenoid or an essential oil.

Suitable examples of biocidal terpenoids can include thymol, carvacrol, eugenol, limonene, and any combination thereof. In one example, the biocidal terpenoid can be carvacrol. Other biocidal terpenoids may be suitable in other embodiments.

Suitable examples of oil compounds (e.g., essential oils) can include tea tree oil, oregano oil, lemon grass oil, lavender oil, citronella oil, eucalyptus oil, clove oil, cinnamon oil, peppermint oil, and any combination thereof. In certain embodiments, a disinfecting composition can include up to about 5% by weight per volume of an oil compound. In certain embodiments, a disinfecting composition can include about 0.5% by weight per volume or less of an oil compound, in certain embodiment, a disinfecting composition can include from about 0.05% to about 0.5% by weight per volume of an oil compound; and in certain embodiments, a disinfecting composition can include from about 0.1% to about 0.3% by weight per volume of an oil compound. Other suitable oil compounds may be used in alternative embodiments, including oil compounds that do not contain a biocidal terpenoid.

The concentration of the surfactant and biocidal terpenoid (or essential oil containing a biocidal terpenoid) present in the antimicrobial agent have a corresponding relationship in certain embodiments. The surfactant may serve as a solubilizing agent for the biocidal terpenoid to render it more effective when present in the disinfecting composition according to one embodiment. In certain embodiments, the concentration of the surfactant and biocidal terpenoid (or source of the biocidal terpenoid) in the antimicrobial agent can have a ratio from about 2:1 to about 1:2. In another embodiment, the ratio can be about 1:1.

In certain embodiments, an antimicrobial agent and/or a disinfecting composition does not include a peroxide (e.g., hydrogen peroxide).

It will be appreciated that the concentrations of some or all of the constituents of the disinfecting composition may be higher or lower than their respective concentrations in a resulting product, such as a hand sanitizer. In various embodiments, the antimicrobial agent may be the disinfecting composition, may be singularly added to form the disinfecting composition or may have various constituents used to form the antimicrobial agent added at different times to help form the resulting disinfecting composition.

The disinfecting compositions contemplated herein can be effective against a multitude of bacterial, fungal or viral strains including at least one of Escherichia coli (ATCC #11229), Escherichia coli (ATCC #35150), Staphylococcus aureus (ATCC #6538), Pseudomonas aeruginosa (ATCC #15442), Bordetella pertussis (ATCC #BAA-589), Trichophyton mentagrophytes (ATCC #9533), Salmonella enterica (ATCC #10708), Influenza A H1N1 (ATCC #VR-1520), methicillin-resistant staphylococcus aureus (MRSA), Enterobacter aerogenes, Saccharomyces cerevisiae (yeast-based strain), Aspergillus, and other species of Staphylococcus. In certain embodiments, the disinfecting compositions can be effective against at least two of Staphylococcus aureus (ATCC #6538), Pseudomonas aeruginosa (ATCC #15442), and Salmonella enterica (ATCC #10708); and in certain embodiments, the disinfecting compositions can be effective against both Staphylococcus aureus (ATCC #6538) and Pseudomonas aeruginosa (ATCC #15442). Other bacterial, fungal or viral strains may be noted in U.S. patent application Ser. No. 14/491,358, filed Sep. 19, 2014, which is hereby incorporated herein by reference.

In certain embodiments, a disinfecting composition can include a bleaching agent (e.g., Clorox®). In certain embodiments, the disinfecting composition can have from about 1% to about 10% by weight per volume of the bleaching agent. In certain embodiments, the disinfecting composition can have from about 1% to about 5% by weight per volume of the bleaching agent. In certain embodiments, the bleaching agent can be chlorine-based. In certain embodiments, the bleaching agent can be peroxide-based. In certain embodiments, the bleaching agent can include sodium hypochlorite, calcium hypochlorite, chlorine dioxide, hydrogen peroxide, sodium percarbonate, sodium perborate, sodium dithionite, sodium borohydride, or combinations thereof. Other suitable bleaching agents are also contemplated.

In certain embodiments, a disinfecting composition can have a pH from about 7 to about 9; and in certain embodiments can have a pH of about 8. The pH of a disinfecting composition can be modified or maintained through inclusion of a buffering agent. Suitable buffering agents can include tris-hydrochloride, tris citrate, and combinations thereof. Other suitable buffering agents may be contemplated. In certain embodiments, a disinfecting composition can have about 10 mM to about 100 mM of a buffering agent. In certain embodiments, a disinfecting composition can from about 25 mM to about 50 mM of a buffering agent.

Application of Disinfecting Compositions with Antimicrobial Agents

The disinfecting compositions described herein are effective in decontaminating and protecting against contamination of surfaces that are susceptible to contamination by microorganisms or formation of biofilm coatings thereon. Effectiveness of the disinfecting compositions can be defined in a variety of ways, including as a percentage of bacterial cells destroyed or killed when subjected to the disinfecting composition. For example, the disinfecting compositions can be at least 99.99% effective against certain bacterial strains such as Staphylococcus aureus (ATCC #6538). This means that for every 1 million Staphylococcus aureus (ATCC #6538) cells subjected to the disinfecting composition all but about 100 Staphylococcus aureus (ATCC #6538) cells remain. In certain embodiments, the disinfecting compositions can be at least 99.999% effective against Staphylococcus aureus (ATCC #6538). In certain embodiments, the disinfecting compositions can be at least 99.9999% effective against Staphylococcus aureus (ATCC #6538). In certain embodiments, the disinfecting compositions can be at least 99.99999% effective against Staphylococcus aureus (ATCC #6538). In certain embodiments, the disinfecting compositions can be at least 99.999999% effective against Staphylococcus aureus (ATCC #6538). For purposes of this disclosure “subjected to” can mean treating a surface having been exposed to a bacterial strain and/or pretreating a surface with the antimicrobial composition that may eventually come into contact with a bacterial strain. A surface can include a variety of objects including tables, beds, countertops, clothing, human skin, medical or exercise equipment and other surfaces that can potentially become contaminated. The disinfecting compositions and antimicrobial agents described herein are generally non-reactive with any such surface. According to certain embodiments, the disinfecting compositions and antimicrobial agents described herein can have little or no toxicity risk, which make them safe for daily use by consumers or medical professionals.

In addition to the general effectiveness of the disinfecting compositions described herein with respect to certain bacterial strains, these disinfecting compositions also remain effective for desired lengths of time, providing the desired persistence needed to protect surfaces. For example, disinfecting compositions described herein may be at least 99.99% effective against Staphylococcus aureus (ATCC #6538) within about 1 minute after being subjected to the disinfecting composition; and in certain embodiments, within about 10 minutes after being subjected to the disinfecting composition. In certain embodiments, the disinfecting composition may be at least 99.99% effective against Staphylococcus aureus (ATCC #6538) about 24 hours after being subjected to the disinfecting composition; in certain embodiments, at least 99.99% effective against Staphylococcus aureus (ATCC #6538) about 48 hours after being subjected to the disinfecting composition; in certain embodiments, at least 99.99% effective against Staphylococcus aureus (ATCC #6538) about 72 hours (3 days) after being subjected to the disinfecting composition; in certain embodiments, at least 99.99% effective against Staphylococcus aureus (ATCC #6538) about 120 hours (5 days) after being subjected to the disinfecting composition; in certain embodiments, at least 99.99% effective against Staphylococcus aureus (ATCC #6538) about 7 days after being subjected to the disinfecting composition; and in certain embodiments, at least 99.99% effective against Staphylococcus aureus (ATCC #6538) about 12 days after being subjected to the disinfecting composition. The lasting or persistent effectiveness of the disinfecting compositions described herein provides substantial benefits to medical institutions in the battle of reducing or eliminating the spread of certain bacterial strains such as MRSA.

The disinfecting compositions described herein may be either ready to use or in concentrated form. The disinfecting compositions can be incorporated into wipes, hard surface cleaners, sanitizers, dishwashing products, including rinse aids, laundry and fabric treatment products, personal or consumer care products such as antiseptics, hand soaps and sanitizers, and lotions to kill or prevent the spread of microorganisms, as well as used in the healthcare and clinical setting as a disinfectant.

While certain disinfecting compositions may provide sufficient effectiveness against one bacteria strain, it was discovered that finding such a composition to be effective against a variety of different bacteria strains can be challenging. The disinfecting compositions described herein however, achieve that result. To better appreciate the effectiveness of the disinfecting compositions described herein the antimicrobial efficacy was measured pursuant to testing performed in accordance with the protocols outlined in AOAC Official Method 961.02 “Germicidal Spray Products as Disinfectants”, as described in AOAC Official Methods of Analysis (2013); and with regard to OCSPP 810.2200 “Disinfectants for Use on Hard Surfaces—Efficacy Data Recommendations (Sep. 4, 2012).”

As is appreciated by a skilled practitioner in the art, the results of the AOAC Germicidal Spray Test indicate the number of test substrates wherein the tested organism remains viable after contact for about 10 minutes with a test disinfecting composition/total number of tested substrates (slides) evaluated in accordance with the AOAC Germicidal Spray Test. Thus, a result of “0/60” indicates that of 60 test substrates bearing the test organism and contacted for about 10 minutes in a test disinfecting composition, 0 test substrates had viable (live) test organisms at the conclusion of the test. A result of 0 test substrates having viable (live) test organisms generally equates to having a reduction of at least 99.9999% of the organism cells that were tested.

The formulations for each of the example disinfecting compositions are provided in Table 1 below. Results of the testing are indicated on the Table 2 below. The reported results indicate the number of test cylinders with live test organisms, number of test cylinders tested for each example formulation and organism tested.

TABLE 1

| Disinfecting Composition | |
|---|---|
| Examples | Formulations |
| Example 1 | 3% (wt./vol.) 1,2-hexanediol |
| | 1.46% (wt./vol.) EDTA |
| | 0.1% (wt./vol.) SDS* |
| | 0.1% (wt./vol.) Carvacrol** |
| | Remainder: deionized water |
| Example 2 | 4% (wt./vol.) 1,2-hexanediol |
| | 1.67% (wt./vol.) EDTA |
| | 0.19% (wt./vol.) SDS* |
| | 0.19% (wt./vol.) Carvacrol** |
| | Remainder: deionized water |

*SDS for each example is sodium dodecyl sulfate (Fisher lot # BKH208).
**Carvacrol for each example is obtained from TCI America.

TABLE 2

| | | | Antimicrobial |
|---|---|---|---|
| | | | Efficacy (as |
| Disinfecting | | | measured under |
| Composition | | Effectiveness (after 10 | AOAC Official |
| Examples | Bacteria Strain Tested | minutes of exposure) | Method 961.02) |
| Example 1 using 3% | Staphylococcus aureus | 100% | PASS |
| 1,2-hexanediol | (ATCC #6538) | | |
| formulation described | Salmonella enterica | 100% | PASS |
| in Table 1 | (ATCC #10708) | | |
| | Pseudomonas | less than 99.9999% | FAIL |
| | aeruginosa (ATCC | | |
| | #15442) | | |
| Example 2 using 4% | Staphylococcus aureus | greater than 99.9999% | PASS |
| 1,2-hexanediol | (ATCC #6538) | | |
| formulation described | Salmonella enterica | greater than 99.9999% | PASS |
| in Table 1 | (ATCC #10708) | | |
| | Pseudomonas | 100% | PASS |
| | aeruginosa (ATCC | | |
| | #15442) | | |

As illustrated in Table 2, the effectiveness and reduction of bacteria strain cells exposed to the formulation of Example 2 was significant. In particular, the formulation of Example 2 when exposed to the Pseudomonas aeruginosa (ATCC #15442) cells achieved an efficacy of greater than 99.9999% (e.g., 100% efficacy) as compared to the formulation of Example 1 when exposed to similar cells, which only had an efficacy of less than 99.9999%. As noted in Table 2, the effectiveness of each of Examples 1 and 2 was measured after 10 minutes of having the respective bacteria strain subject to the respective compositions. In addition to the improvement seen in the effectiveness and reduction of the bacteria strain cells, Example 2 proved more effective than Example 1 when evaluated under AOAC Official Method 961.02. To be deemed effective (i.e., to receive a score of “PASS”) against any of the three bacteria strains tested in Table 2 with respect to AOAC Official Method 961.02, a tested disinfecting composition must produce complete kill of the respective bacteria for at least 59 out of 60 cells. For those disinfecting compositions that met this requirement they received a designation of “PASS”, and for those that did not received a designation of “FAIL”. With respect to the antimicrobial efficacy for Example 1, under AOAC Official Method 961.02, that disinfecting composition received a “PASS” for both Staphylococcus aureus (ATCC #6538) and Salmonella enterica (ATCC #10708), but a “FAIL” for Pseudomonas aeruginosa (ATCC #15442). With respect to the antimicrobial efficacy for Example 2, under AOAC Official Method 961.02, that disinfecting composition receives a “PASS” for each of Staphylococcus aureus (ATCC #6538), Salmonella enterica (ATCC #10708), and Pseudomonas aeruginosa (ATCC #15442).

In addition to having enhanced efficacy against bacteria strains, the disinfecting compositions described herein in a further embodiment are effective against enveloped viruses, such as Influenza A H1N1 (ATCC #VR-1520). To determine the effectiveness of the disinfecting compositions against Influenza A H1N1 (ATCC #VR-1520) the protocols outlined in the American Society for Test Materials (ASTM) E1053-11 and Standard Test Method to Assess Virucidal Activity of Chemicals Intended for Disinfection of Inanimate, Nonporous Environmental Surface, with modifications based on U.S. Environmental Description of Test Substance and the Official Methods of Analysis, 961.02, AOAC Germicidal Spray Products as Disinfectants can be used. For a formulation similar to that in Table 1 for Example 2, where the concentration of 1,2-hexanediol is 4.25% instead of 4%, the percent reduction of the Influenza A H1N1 virus would be at least 99.99% for example.

As may be seen from the results indicated above, the improved disinfecting compositions according to the various embodiments provide excellent antimicrobial efficacy against known bacteria commonly found in bathrooms, kitchens, hospitals, clinics, and other environments, as well as superior safety and persistence effects compared to traditional and existing antimicrobial agents.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

What is claimed is:

1. A disinfecting composition comprising an antimicrobial agent, the composition comprising:
a) from about 1.5% to about 2.0% by weight per volume of an ethylenediamine tetraacetic acid or salts thereof (EDTA);
b) from about 4% to about 4.5% by weight per volume of a straight chain 1,2-alkanediol, wherein the straight chain 1,2-alkanediol comprises 1,2-hexanediol;
c) from about 0.15% to about 0.5% by weight per volume of a surfactant; and
d) from about 0.15% to about 0.5% by weight per volume of one of a biocidal terpenoid or an essential oil; and
e) water.

2. The disinfecting composition of claim 1, the composition exhibiting antimicrobial efficacy against each of Staphylococcus aureus (ATCC #6538) and Pseudomonas aeruginosa (ATCC #15442) in accordance with the protocols of AOAC Official Method 961.02.

3. The disinfecting composition of claim 1, wherein the surfactant is about 0.2% by weight per volume.

4. The disinfecting composition of claim 1, wherein the one of the biocidal terpenoid or the essential oil is about 0.2% by weight per volume.

5. The disinfecting composition of claim 1, wherein the ratio of the concentration of the surfactant to the one of the biocidal terpenoid or the essential oil is about 2:1 to about 1:2.

6. The disinfecting composition of claim 1, wherein the surfactant comprises a cationic salt of dodecyl sulfate.

7. The disinfecting composition of claim 1 having a pH from about 7 to about 9.

8. The disinfecting composition of claim 1, wherein the one of the biocidal terpenoid or the essential oil comprises at least one of thymol, carvacrol, eugenol, and limonene.

9. The disinfecting composition of claim 8, wherein the one of the biocidal terpenoid or the essential oil comprises carvacrol.

10. The disinfecting composition of claim 1 further exhibiting antimicrobial efficacy of at least 99.99% against Influenza A H1N1 in accordance with the protocols of the American Society for Test Materials (ASTM) E1053-11.

11. The disinfecting composition of claim 1 being at least 99.99% effective against Staphylococcus aureus (ATCC #6538) after 10 minutes of being applied to a surface.

12. The disinfecting composition of claim 1 being at least 99.99% effective against Staphylococcus aureus (ATCC #6538) after 24 hours of being applied to a surface.

13. The disinfecting composition of claim 1 being at least 99.99% effective against Staphylococcus aureus (ATCC #6538) up to 5 days after being applied to a surface.

14. The disinfecting composition of claim 1 being at least 99.99% effective against Staphylococcus aureus (ATCC #6538) up to 12 days after being applied to a surface.

15. A personal care product comprising the disinfecting composition of claim 1.

16. A clinical disinfectant product comprising the disinfecting composition of claim 1

17. The disinfecting composition of claim 1 further exhibiting antimicrobial efficacy against at least one of Salmonella enterica (ATCC #10708) and Escherichia coli (ATCC #11229) in accordance with AOAC Official Method 961.02.

18. The disinfecting composition of claim 1 being non-reactive with a surface on which it is applied.

19. A method of treating a surface, the method comprising applying an effective amount of a disinfecting composition of claim 1 to a surface in need of treatment.

20. A method of preventing contamination of a surface with a bacteria, fungi, or virus, the method comprising applying the disinfecting composition of claim 1 to a surface.
